(19) United States
(12) Patent Application Publication
(10) Pub. No.: US 2019/0276387 A1
(43) Pub. Date: Sep. 12, 2019

(54) PRECURSOR COMPOUNDS FOR FRAGRANT ALDEHYDES

(71) Applicant: GIVAUDAN SA, Vernier (CH)

(72) Inventors: Felix FLACHSMANN, Duebendorf (CH); Nathalie JOSET, Duebendorf (CH); Martin Alan LOVCHIK, Duebendorf (CH); Veronika ZELENAY, Duebendorf (CH)

(21) Appl. No.: 16/347,289

(22) PCT Filed: Nov. 28, 2017
(86) PCT No.: PCT/EP2017/080686

§ 371 Date: May 3, 2019

(30) Foreign Application Priority Data
Nov. 28, 2016 (GB) 1620044.6

(51) Int. Cl. C07C 69/738; C07C 69/593; C07C 255/23; C07C 67/333

(52) U.S. Cl. CPC C07C 69/738; C07C 69/593; C11D 3/507; C07C 67/333; C07C 255/23

(57) ABSTRACT

A precursor compound of a fragrant aldehyde is provided, as well as a method of forming such a compound, a kit comprising said compound, and certain uses thereof. The precursor compound has formula (I),

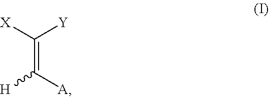

wherein A is a hydrocarbon residue of a fragrant aldehyde A-CHO; and X and Y are independently selected from the group consisting of a nitrile, a keto, and an ester functional group. One such precursor compound is ethyl 2-acetyl-4-methyltridec-2-enoate enriched in its Z-isomer, which is a thermally stable precursor of 2-methyl undecanal.

### FIELD OF THE INVENTION

This invention is concerned with compounds, compositions and methods useful in the generation of fragrant aldehydes.

### BACKGROUND TO THE INVENTION

The provision of fragrance ingredients by means of a fragrance precursor compound, which is not itself considered useful as a fragrance ingredient, but under certain conditions, such as exposure to light, heat, pH change or enzymatic activity will break down to provide one or more fragrant compound(s), is known in the art.

An example of such precursor compounds can be found in the class of di-carbonyl alkylidene compounds described in WO 2007/143873 A1, having the general structure

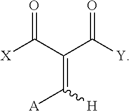

The alkylidene group in conjugation with the carbonyl groups is labile and will hydrolyze to release one or more aldehyde(s) A-CHO. As such, the compounds described in WO 2007/143873 A1 potentially offer a means for providing fragrant aldehydes.

However, whilst investigating these compounds, applicant found that they are susceptible to deactivation over relatively short periods of time, rendering the deactivated material incapable of acting as a precursor compound for the release of the fragrant aldehyde.

The provision of fragrant aldehyde precursor compounds, which are capable of releasing a fragrant aldehyde when needed in application, but which are also stable over prolonged conditions of storage; methods of selecting such precursor compounds; as well as methods of preparing and storing said precursor compounds, remain unmet needs in the art.

### SUMMARY OF THE INVENTION

Investigating prior art precursor compounds, such as those compounds disclosed in WO 2007/143873 A1, applicant found that the conjugated alkylidene double bond is susceptible to migration in a non-reversible process, resulting in deactivation of the precursor compounds.

The present invention addresses the problems of the prior art and provides in a first aspect a precursor compound for a fragrant aldehyde according to the formula (I)

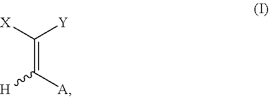

wherein

A is a hydrocarbon residue of a fragrant aldehyde A-CHO, wherein the hydrocarbon residue may optionally contain one or more hetero-atom(s) selected from O, N, S and/or Si;

X and Y are independently selected from the group consisting of a nitrile (—CN), a keto (—COR) or an ester (—CO₂R′) functional group, wherein R and R′ are independently an alkyl residue, and more particularly methyl, ethyl, propyl, isopropyl, butyl, sec-butyl, iso-butyl and pentyl, provided that:
i) X and Y cannot both represent keto residues; and
ii) when X and Y represent different functional groups, wherein one group is an ester group and the other one is a keto group, the alkylidene double bond is enriched in its Z-isomer.

In another aspect of the invention, there is provided a method of preventing or reducing the extent and/or rate of deactivation of a fragrant aldehyde precursor compound according to formula (I), said method comprising the step of selecting the substituents X and Y hereinabove defined, such that
i) if X and Y are the same, they are not keto groups; and
ii) if X and Y represent different functional groups, wherein one group is an ester group and the other one is a keto group, the alkylidene double bond is enriched in its Z-isomer.

In another aspect of the invention, there is provided a method of forming the fragrant aldehyde precursor compounds according to the formula (I) as defined above, comprising the step of condensing a compound of the structure X—(CH)₂—Y, wherein X and Y are as defined above, with a fragrant aldehyde A-CHO in the presence of a base, under conditions in which either X and Y are not both represented by the keto-group. This allows for preventing or reducing the extent and/or rate of deactivation of the fragrant aldehyde precursor compounds (I). To this end, when X and Y represent different functional groups, wherein one group is an ester group and the other one is a keto group, the condensation reaction is carried out under conditions that favour formation of the alkylidene double bond in its Z-configuration.

Preferably, said fragrant aldehyde precursor compound (I) is stored at a temperature of about 20° C. or less, and more particularly of about 10° C. or less, in order to prevent or reduce the extent and/or rate of deactivation. This allows for a storage of at least one year.

In another aspect of the invention, there is provided a kit comprising the fragrant aldehyde precursor compound (I) in a container, together with instructions to store the container containing said compound at a temperature of about 20° C. or less, and more particularly of about 10° C. or less.

In still another aspect of the invention, there is provided a fragrant aldehyde precursor compound of the formula (I) in a composition comprising at least one surfactant.

These and other aspects of the invention will be more fully described by reference to the following detailed description and examples.

### DETAILED DESCRIPTION OF THE INVENTION

The genus of compounds disclosed in WO 2007/143873 A1 represents a potentially useful precursor means for the generation of fragrant aldehydes. However, the applicant surprisingly found manner that these compounds are in fact susceptible to deactivation. More specifically, the applicant found that the alkylidene double bond can shift (isomerize) to form an ethylenic double bond that is no longer conjugated with the carbonyl double bond. As a result, the double bond shifted isomers cannot degrade hydrolytically to release a fragrant aldehyde. Furthermore, the shift of the double-bond was found to be irreversible.

Surprisingly, however, the applicant found that the nature of the residues X and Y influence the propensity towards double-bond isomerization, and that the appropriate selection of X and Y functional groups can prevent or reduce substantially the rate and/or extent of double-bond shifting and thus deactivation. Furthermore, applicant found that even in the case of compounds that are susceptible to deactivation, methods of synthesis and storage of the compounds can substantially reduce the rate and/or extent of deactivation.

According to a first finding, the rate and/or extent of isomerization can be reduced or even prevented through the appropriate selection of X and Y residues. In a particular, when the functional groups represented by X and Y are the same, they should not be keto groups.

A particular class of fragrant aldehyde precursor compounds is represented by the malonate ester according to the following general formula

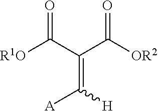

wherein R¹ and R² can be the same or different, each being an alkyl residue, and more particularly methyl, ethyl, propyl, isopropyl, butyl, sec-butyl, iso-butyl and pentyl; and A is as hereinabove defined.

Another particular class of a fragrant aldehyde precursor compound is the beta-keto ester according to the following general formula

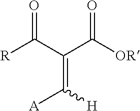

wherein R, R′, and A are as hereinabove defined, and the alkylidene double bond is enriched in its Z-isomer.

Particularly preferred precursor compounds are those having the following formula (II)

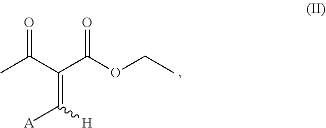

wherein the akylidene double bond is enriched in its Z-isomer. Preferably, the ratio of the Z- to the E-isomer is 55:45 to 100:0, more preferably at least 60:40, 70:30 or even 80:20. The Z-enriched compound of formula (II) may be obtained directly by selective synthesis or may be obtained by purification from a mixture, e.g. by column chromatography.

Still another particular class of a fragrant aldehyde precursor compounds is the cyanoacetate according to the following general formula

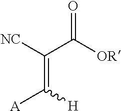

wherein R′ and A are as hereinabove defined.

The fragrant aldehyde A-CHO may be any fragrant aldehyde useful in perfumery as a perfumery ingredient. They are well-known to the person skilled in the art.

Examples of fragrant aldehydes from which the moiety A may be derived include, but are not limited to, the following: 2,6,10-trimethylundec-9-enal; 8,8-dimethyl-1,2,3,4,5,6,7,8-octahydronaphthalene-2-carbaldehyde; (4-isopropyl-phenyl)-ethanal; 2,4-dimethyl-cyclohex-3-ene-1-carbaldehyde; 1,3,5-trimethyl-cyclohex-1-ene-4-carbaldehyde; 4-(4-hydroxy-4-methylpentyl)cyclohex-3-ene-1-carbaldehyde; hex-2-enal; 3,5,5-trimethyl-hexanal; heptanal; 2,6-dimethylhept-5-enal; decanal; dec-8-enal; dec-9-enal; dec-4-en-1-al; 2-methyl-decanal; undec-10-en-1-al; undecanal; dodecanal; 2-methyl-undecanal; tridecanal; tridec-2-enal; octanal; nonanal; non-2-enal; (6Z)-nonenal; undec-9-enal; 2-phenyl-propanal; 2-(4-methyl-phenyl)-ethanal; 3,7-dimethyloctanal; dihydrofarnesal; 7-hydroxy-3,7-dimethyl-octanal; 2,6-dimethyl-oct-5-en-1-al; 3-(3-isopropyl-phenyl)-butanal (Florhydral); 2-(3,7-dimethyl-oct-6-en-oxy)-ethanal; 4-(4-methylpent-3-enyl)-cyclohex-3-ene-1-carbaldehyde; 2,3,5,5,-tetramethyl-hexanal; longifolic aldehyde; 2-methyl-4-(2,6,6-trimethylcyclohex-2-en-1-yl)-butanal; 2-methyl-3-(4-tert-butylphenyl)propanal (Lilial); 3-(4-tert-butyl-phenyl)-propanal; 2-(4-isopropyl-phenyl)-propanal; 3-(4-isobutylphenyl)-propanal; 3-(4-isobutyl-2-methylphenyl)propanal (Nympheal); 3-(benzo[1,3]dioxol-5-yl)-2-methyl-propanal; 3,7-dimethyl-oct-6-ene-1-al; 2-methyl-3-(4-isopropylphenyl)-propanal; 4-tert-butyl-cyclohexane-1-carbaldehyde; 2,6,6-trimethyl-1,3-cyclohexadiene-1-carboxaldehyde (Safranal); 4-(octahydro-4,7-methano-5H-inden-5-ylidene)butanal (Dupical); (3,7-dimethyl-oct-6-enyloxy)-ethanal; (2E,6Z)-nonadienal; 2,4-dimethyl-2,6-heptadienal; (E)-dec-2-enal; dodec-2-enal; 3,7-dimethyl-octa-2,6-dienal; 2,4-diethyl-hepta-2,6-dienal; 3,7-dimethyl-nona-2,6-dienal; 3-propyl-hept-2-enal; 2,6-dimethyl-5-heptenal (Melonal); and 4-isopropenyl-cyclohex-1-ene-1-carbaldehyde (Shisolia).

Particularly preferred examples of fragrant aldehydes as set forth herein-below:

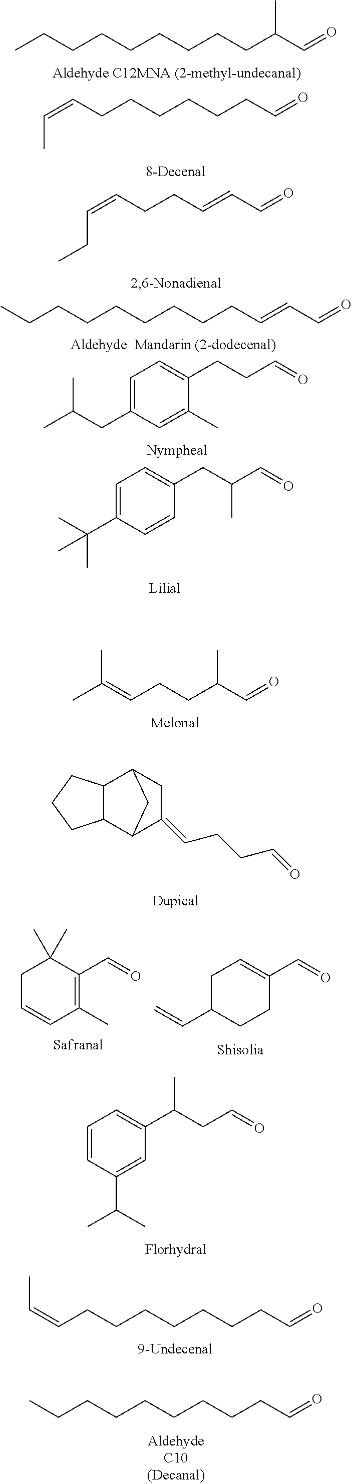

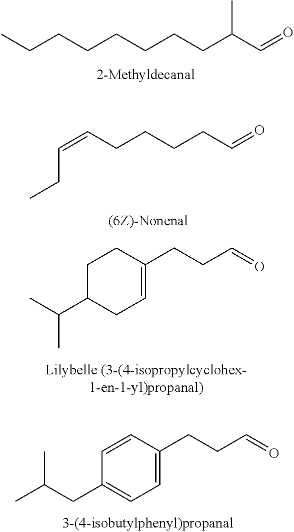

Compounds of formula (I) that are precursors of the aforementioned fragrant aldehydes form particular embodiments of the present invention.

A method of forming the aforementioned fragrant aldehyde by means of the hydrolytic degradation of precursor compounds according to the formula (I) forms yet additional aspect of the present invention.

A particular class of precursor compounds according to the invention is able to degrade and thereby release the fragrant aldehyde 2-methyl-undecanal.

A more particular class of a precursor compound is the beta-keto-ester referred to hereinabove, enriched in its Z-isomer, which is able to degrade and release the fragrant aldehyde 2-methylundecanal.

Still more particularly, a precursor compound according to the present invention is a beta-keto-ester, as hereinabove defined, enriched in its Z-isomer, that is able to degrade and release the fragrant aldehyde 2-methyl-undecanal, and wherein R attached to the keto-carbonyl carbon atom is a methyl residue, and R′ attached to the ester carbonyl carbon atom is an ethyl residue, i.e. ethyl 2-acetyl-4-methyltridec-2-enoate (III)

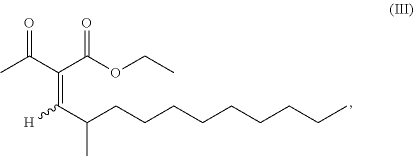 [SMILES: CCCCCCCCCC(C)C=C(C(C)=O)C(=O)OCC]

wherein the alkylidene double bond is enriched in its Z-isomer. Preferably, the ratio of the Z- to the E-isomer is 55:45 to 100:0, more preferably at least 60:40, 70:30 or even 80:20.

According to a second finding, an additional or alternative means of preventing or reducing the rate and/or extent of isomerization is through the careful selection of the reaction conditions employed in the synthesis of the compounds of formula (I), and/or the conditions under which these compounds are stored.

Compounds of the formula (I) may be formed by a Knoevenagel condensation reaction, between a fragrant aldehyde A-CHO and a compound X—(CH)₂—Y in the presence of a base, wherein the groups X, Y and A are as hereinabove defined:

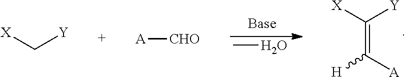

The base may be selected from cyclic secondary amines, such as piperidine, piperazine or pyrrolidine, linear primary or secondary mono- or diamines, such as dodecylamine, β-amino isobutanol or ethylene diamine, amino acids, such as L-pyrrolidine or β-alanine, aromatic amines, such as aniline, polymeric amines, such as polyethylene imines known under the name of Lupasol™, or tertiary amines in the presence of Lewis acids, such as pyridine in the presence of TiCl₄. The amines may be used in quantities of 0.001-10 mol %, in free forms or as mixtures with acids, such as acetic acid or sulfamic acid, wherein the acid may be admixed in ratios up to equi-molar amounts with respect to the amine. The catalytic amine may also be used on solid support, for example polystyrene bound piperazine crosslinked with divinylbenzene, Aldrich catalogue No 526290 (200-400 mesh, 1-2 mmol/g active amine), or Amberlyst® A21 free base, Aldrich catalogue No 216410, or the amine catalyst may be immobilized on silica gel, as for example in 3-(1-Piperazino)propyl functionalized silica gel, Aldrich catalogue No 552607 (200-400 mesh, 0.8 mmol/g active amine).

The Knoevenagel condensation reaction is carried out preferentially at temperatures between 0° C. and 60° C. in a pH range between 5-8. The reaction can be run at ambient pressure or under reduced pressure, i.e. 0.01-100 mbar, which will facilitate the removal of water formed as a byproduct either by distillation or via a flow of air or nitrogen through the reaction compartment.

Although it is stated in WO 2007/143873 A1 that the nature of the substituents on the 1,3-dicarbonyl compound plays no part in the reaction, and may be selected from a wide variety of materials, applicant found that this is not in fact the case. More particularly, the nature of the functional groups X and Y has a profound effect on the reaction and the ultimate effectiveness of the compounds as fragrant aldehyde precursors.

In order to illustrate this, applicant refers to a particular compound of the present invention—ethyl 2-acetyl-4-methyltridec-2-enoate—which may be formed in a process according to the present invention as a mixture of its Z- and E-isomers

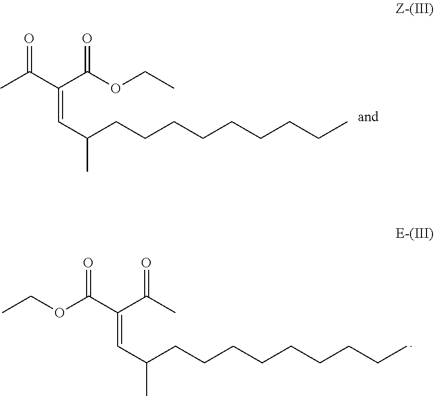

The applicant found that from a mixture of both the E- and the Z-isomers, the formation of a double bond shifted product (IV) is observed, which is inactive as a fragrant aldehyde precursor:

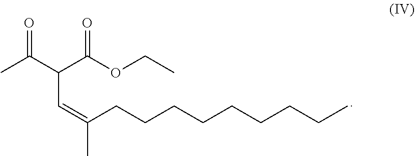 [SMILES: CCCCCCCCC/C(C)=C\C(C(C)=O)C(=O)OCC]

The applicant surprisingly found that the E-isomer was far more susceptible to this process and it would isomerize more quickly and to a greater extent compared with the Z-isomer.

Accordingly, the invention provides in another of its aspects a compound of formula (I), which is free or is substantially free of its double-bond shifted isomer. In particular, the invention provides a compound of formula (I) in admixture with less than 50% of its double-bond shifted isomer, preferably less than 30%, more preferably less than 10%, and most preferably at least essentially free of its double-bond shifted isomer.

In an embodiment of the invention, the compound of formula (I) is ethyl 2-acetyl-4-methyltridec-2-enoate.

As used herein, when a compound of formula (I) is said to be enriched in its Z-isomer, this refers to a compound in the form of a mixture of both E- and Z-isomers in which the mixture contains more Z-isomer than E-isomer, more preferably, the Z-isomer in pure form. More particularly, a compound of formula (I) is enriched in its Z-isomer when the Z:E ratio is in the range of 55:45 to 100:0, more preferably at least 60:40, 70:30 or even 80:20.

As used herein, when a compound of formula (I) is referred to as being free or substantially free of its double-bond shifted isomer, it means that the double-bond-shifted isomer is undetectable in a sample of a compound of formula (I), or the double-bond shifted isomer exists in admixture with a compound of formula (I) in an amount of up to 50% wt/wt, and more particularly 1 to 50% wt/wt.

Double-bond-shifted compounds, including the double-bond-shifted isomer (IV) of ethyl 2-acetyl-4-methyltridec-2-enoate (III), are novel compounds, and these compounds, and their mixtures with E- and/or Z-isomers of the compounds of formula (I) form additional aspects of the invention.

In another aspect of the present invention, there is provided a method of preparing a compound of formula (I) as hereinabove defined, particularly a keto-ester compound of formula (I), said method comprising the selection of starting materials and reaction conditions that promote the enrichment of the Z-isomer.

As stated hereinabove, compounds and mixtures of the present invention can be prepared by a Knoevenagel Condensation reaction. If a compound of formula (I) that is enriched in its Z-isomer is required, as is the case when the compound of formula (I) is a keto-ester, the use of low amounts of an amine catalyst, e.g. <1 mol % or even more preferred <0.1 mol % as well as low temperatures, e.g. <80° C., more preferred <60° C., favour product mixtures containing more Z-isomer than E-isomer. Alternatively, the use of appropriate inorganic catalysts, such as calcium carbonate, may lead to a higher Z:E ratio. The formation of product mixtures with high Z contents is also promoted by running the reaction in a pH range of 5-8.

In the selection of reaction conditions that favour the formation of high levels of Z-isomer, the skilled person will appreciate that it might be uneconomical to synthesize a compound of formula (I) that is very highly enriched in the Z-isomer. Indeed, the skilled person might consider it advantageous to form a mixture of Z- and E-isomers and then enrich, or further enrich the mixture in the Z-isomer using separation methods such as chromatography, distillation or membrane filtration.

Thus, a compound characterized by a high Z-isomer content can also be obtained by employing chromatographic techniques, more particular silica gel chromatography, and more particularly still silica gel chromatography using a mixture of hexane and tert-butyl methyl ether in a 4:1 to 9:1 ratio.

Thus, in accordance with the present invention, compounds of formula (I) that are enriched in the Z-isomer, may be prepared synthetically, or by a combination of synthetic and purification steps.

In accordance with the present invention, compounds of the formula (I) in which X and Y represent different functional groups, (e.g. the keto-esters referred to above) may be obtained as mixtures of their Z- and E-isomers. Furthermore, given that the E-isomer is susceptible to relatively rapid deactivation, it may be highly desirable to prevent or reduce the extent and/or rate of Z- to E-isomerization.

Accordingly, the invention provides in another of its aspects a method of preventing Z- to E-isomerization; or reducing the extent of Z- to E-isomerization; or reducing the rate of Z- to E-isomerization, in a compound of formula (I) presented in the form of a mixture of its Z- and E-isomers, or in its pure Z-isomer form, said method comprising the step of storing the compound of formula (I) under conditions that do not favour Z- to E-isomerization.

Applicant surprisingly found that residues of the base catalyzing the process of forming compounds of formula (I) as well as residues of the derivatives of said base can promote Z- to E-isomerization.

Accordingly, in a preferred embodiment, the method of the present invention comprises the step of removing residual base and/or residual derivatives of the base. This is preferably done once the Knoevenagel condensation reaction is at least essentially completed, e.g. has run to a conversion of at least 70%, preferably at least 80%, more preferably at least 90%. By removing residual base and/or residual derivatives of the base, it is possible to prevent Z- to E-isomerization, or to reduce the extent of Z- to E-isomerization; or to reduce the rate of Z- to E-isomerization in a compound of formula (I) presented in the form of a mixture of its Z- and E-isomers, or in its pure Z-isomer form.

The invention also provides in another aspect a compound of formula (I) that is free or is substantially free of residual base, and/or residual derivatives of said base.

A compound of formula (I) that is isolated from, and is free or is substantially free of residual base or residual derivatives of said base, preferably contains less than 0.1 wt %, and more particularly less than 0.01 wt % of residual base, or residual derivatives of said base, based on the weight of the compound of formula (I).

Bases that are particularly effective in catalyzing Z- to E-isomerization are amines, such as piperidine and piperazine, and it is particularly desirable to isolate compounds of formula (I) when such bases are employed in the preparation of these compounds. Isolation of residual bases or their derivatives can be effected by known isolation techniques, such as chromatography, distillation or filtration.

In order to facilitate separation of catalyst from a compound of formula (I), it may be advantageous to employ a catalyst on a solid support, such as polystyrene bound piperazine crosslinked with divinylbenzene, Aldrich catalogue No 526290 (200-400 mesh, 1-2 mmol/g active amine), or Amberlyst® A21 free base, Aldrich catalogue No 216410, or the amine catalyst may be immobilized on silica gel, as for example in 3-(1-piperazino)propyl functionalized silica gel, Aldrich catalogue No 552607 (200-400 mesh, 0.8 mmol/g active amine), which can be removed from the reaction mixture after formation of a compound of formula (I) by filtration.

Furthermore, the applicant surprisingly found that residual unreacted fragrant aldehyde A-CHO can promote Z- to E-isomerization in a compound of formula (I).

Accordingly, the invention provides in another of its aspects, method of preventing Z- to E-isomerization; or reducing the extent of Z- to E-isomerization; or reducing the rate of Z- to E-isomerization in a compound of formula (I) presented in the form of a mixture of its Z- and E-isomers, or as the pure Z-isomer, said method comprising the step of isolating the compound of formula (I) from residual unreacted fragrant aldehyde A-CHO.

Therefore, in a preferred embodiment, the method of the present invention comprises the step of removing residual unreacted 2-methyl-undecanal from the reaction mixture. This is preferably done once the Knoevenagel condensation reaction is at least essentially completed, e.g. has run to a conversion of at least 70%, preferably at least 80%, more preferably at least 90%. By removing residual unreacted 2-methyl-undecanal from the reaction mixture, it is possible to of prevent Z- to E-isomerization; or to reduce the extent of Z- to E-isomerization; or to reduce the rate of Z- to E-isomerization in a compound of formula (I) presented in the form of a mixture of its Z- and E-isomers, or as the pure Z-isomer.

The invention also provides in another aspect a compound of formula (I) that is free or is substantially free of any residual unreacted fragrant aldehyde A-CHO.

A compound of formula (I) that is isolated from, and is free or is substantially free of residual unreacted fragrant aldehyde preferably contains less than 10.0 wt %, and more particularly less than 1.0 wt % and more particularly still less than 0.1 wt % of residual unreacted fragrant aldehyde A-CHO.

Removal of residual unreacted fragrant aldehyde can be effected by known isolation techniques, such as chromatography, distillation or any other suitable techniques known to the person skilled in the art.

The applicant also surprisingly found that Z- to E-isomerization could be prevented; or the extent of isomerization reduced; or the rate of isomerization reduced by storing a compound of formula (I) presented in the form of a mixture of its Z- and E-isomers, or as the pure Z-isomer at a temperature of 20° C. or less, preferred about 6° C. or less.

Accordingly, the invention provides in yet another of its aspects a method of preventing Z- to E-isomerization; or reducing the extent of Z- to E-isomerization; or reducing the rate of Z- to E-isomerization, in a compound of formula (I) presented in the form of a mixture of its Z- and E-isomers; or as its pure Z-isomer, said method comprising the step of storing the compound of formula (I) at a temperature of about 20° C. or less, preferred about 6° C. or less.

In yet another aspect of the invention, there is provided a kit comprising a container containing a compound of formula (I) together with labeling, instructions and/or packaging providing instructions to store the compound at a temperature of about 20° C. or less, preferred about 6° C. or less.

Compounds of formula (I) may be employed in all manner of fine and technical perfumery applications in which it is desired to provide the odour of a fragrant aldehyde, wherein the release of the odour is desired at some particular point in time and is caused by the presence of moisture. Applications include, but are not limited to, laundry care products, such as products employed in a wash liquor, in a dryer or on laundry post-drying; personal care products, such as hair products, skin products and cosmetics; and household care products, such as hard-surface cleaners and air-care products.

The compounds of formula (I) are particularly useful in perfumery applications in which they are employed during the wet stage of the application, such as being added as a component of a detergent or conditioner composition to a washing liquor; or as a component of a shampoo, hair conditioner, or body wash, body cream or body lotion to the skin or hair of a human subject, and to release a lasting odour of a fragrant aldehyde from a drying or dry situs (such as fabric and human skin or hair) onto which the compound is applied.

Accordingly, the invention provides in another of its aspects a method of imparting the fresh odour of a fragrant aldehyde A-CHO to a dry or drying situs, such as a fabric, a household surface, or the skin or hair of a human subject, said method comprising the steps of treating the situs with, respectively, a laundry care composition, a personal care composition, or a household care composition containing said compound of formula (I), and allowing the situs to dry.

In a particular embodiment of the present invention, there is provided a method of imparting the fresh odour of a fragrant aldehyde A-CHO to a dry or drying fabric, said method comprising the step of washing or treating the fabric with the laundry care composition, preferably in an aqueous liquor containing a detergent or fabric conditioner or fabric treatment composition containing said compound (I), and thereafter allowing the fabric to dry.

In another particular embodiment, there is provided a method of imparting a fresh odour of a fragrant aldehyde A-CHO to drying or dry human skin or hair, said method comprising the step of washing or treating the human hair or skin with the personal care composition, in particular a hair care, body care, or cosmetic product, and thereafter allowing the hair or skin to dry.

In yet another particular embodiment, there is provided a method of imparting a fresh odour of a fragrant aldehyde A-CHO to a drying or dry household surface, said method comprising the step of applying a household surface cleaner or treatment composition to the household surface, and allowing the surface to dry.

Laundry care, personal care and household care compositions comprising a compound of formula (I) form yet another aspect of the present invention.

Furthermore, applicant surprisingly found that the presence of surfactants contained in laundry care, personal care and household care products can prevent Z- to E-isomerization; reduce the extent of Z- to E-isomerization; or reduce the rate of Z- to E-isomerization.

Accordingly, the invention provides in yet another of its aspects a method of preventing Z- to E-isomerization; reducing the extent of Z- to E-isomerization; or reducing the rate of Z- to E-isomerization of a compound of formula (I) presented in the form of a mixture of its Z- and E-isomers, or in pure Z-isomer form, said method comprising the step of dispersing a compound of formula (I) into a laundry care product, a personal care product, or a household care product containing at least one surfactant. Thus, the present invention also encompasses a laundry care product, a personal care product, or a household care product containing a compound of formula (I) and at least one surfactant.

When employed in the manner described herein above, the compounds of formula (I) may be combined with any other perfumery ingredients commonly used in the art. The compounds of formula (I) are useful complements in perfume compositions that are typically employed in all manner of fine and technical perfumery. By using compounds of formula (I), it is possible to impart to a perfume composition the floral-fresh impact of aldehyde perfumery ingredients, but in a long-lasting manner. This is particularly desirable because aldehyde perfumery ingredients are usually impactful, but they are not substantive and are short-lasting. In this way, precursor compounds of the present invention are useful alternatives of free-aldehyde ingredients when long-lasting freshness on dry stages of application are required, because to obtain the floral-fresh impact of aldehyde ingredients during dry-down or on dry stages of application would require the use of very high concentrations of free-aldehyde in perfume compositions, which would be perceived as too harsh and unbalanced during the wet stages of application.

There now follows a series of examples serving to further illustrate the invention.

### Example 1: Preparation of rac. ethyl 2-acetyl-4-methyltridec-2-enoate (III)

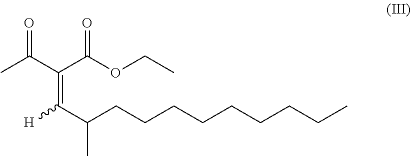 [SMILES: CCCCCCCCCC(C)C=C(C(C)=O)C(=O)OCC]

Ethyl acetoacetate (239.8 g, 1.83 mol, 1.2 equiv.) and 2-methyl undecanal (“Aldehyde C12MNA”, 283.0 g, 1.52 mol, 1.0 equiv.) were placed in a 1.5 L glass reaction flask equipped with a mechanical stirrer and a distillation apparatus (15 cm Vigreux column) connected to a vacuum outlet. Piperidine (517 mg, 6.0 mmol, 0.4 mol %) was added and the mixture was stirred at 50° C. and ambient pressure for 1 h, after which time the mixture became turbid. Vacuum was applied (100 mbar) and stirring at 50° C. was continued for 24 h. The distillation receiver flask was cooled with an ice bath. A total of 59 g of distillate was recovered, containing water and ethyl acetoacetate. The apparatus was brought to ambient pressure and cooled to room temperature. The product (III) (426 g, 95%) was obtained as a clear, pale yellow oil exhibiting an aldehydic fruity-orange scent. The product was composed of 36% (E)-2-acetyl-4-methyltridec-2-enoate, 58% (Z)-2-acetyl-4-methyltridec-2-enoate, 5% ethyl 2-acetyl-4-methyltridec-3-enoate and 0.8% of 2-methyl undecanal (NMR analysis with internal standard p-methoxy benzaldehyde).

### Example 2: Preparation of pure Z- and E-Isomers of rac. ethyl 2-acetyl-4-methyltridec-2-enoate (III)

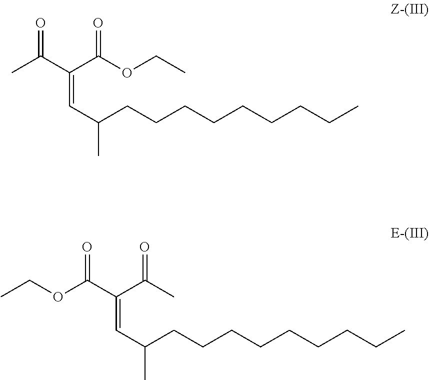

Pure E- and Z-isomers of ethyl 2-acetyl-4-methyltridec-2-enoate (III) were obtained by silica gel column chromatography of 25 g of the crude product obtained from the condensation of ethyl acetoacetate and 2-methyl undecanal as described in Example 1 with hexane/MTBE 4:1 as eluent.

From this, 4.3 g (17%) of pure ethyl (Z)-2-acetyl-4-methyltridec-2-enoate Z-(III) was obtained and 3.2 g (13%) of 95% pure ethyl (E)-2-acetyl-4-methyltridec-2-enoate E-(III), which was further purified by a second silica gel column chromatography with hexane/MTBE 9:1 to obtain 2.5 g of pure ethyl (E)-2-acetyl-4-methyltridec-2-enoate E-(III). Both products were colourless oils exhibiting an aldehydic fruity-orange scent.

Ethyl (E)-2-acetyl-4-methyltridec-2-enoate E-(III):

R_{f} (hexane/MTBE 4.1)=0.60

¹H-NMR (400 MHz, C₆D₆) 6.75 (d, J=10.8 Hz, 1H), 3.93 (q, J=7.1 Hz, 2H), 2.45-2.61 (m, 1H), 2.21 (s, 3H), 1.09-1.28 (m, 16H), 0.89 (t, J=7.3 Hz, 3H), 0.89 (t, J=6.9 Hz, 3H), 0.86 (d, J=6.6 Hz, 3H).

Ethyl (Z)-2-acetyl-4-methyltridec-2-enoate Z-(III):

R_{f} (hexane/MTBE 4.1)=0.47

¹H-NMR (400 MHz, C₆D₆) 6.42 (d, J=10.5 Hz, 1H), 4.06 (q, J=7.1 Hz, 2H), 2.62 (m, J=10.5, 6.5 Hz, 1H), 1.94 (s, 3H), 1.14-1.37 (m, 16H), 0.99 (t, J=7.1 Hz, 3H), 0.91 (d, J=6.8 Hz, 3H), 0.90 (t, J=7.0 Hz, 3H).

### Example 3: Preparation of ethyl 2-acetyl-4-methyltridec-3-enoate (IV)

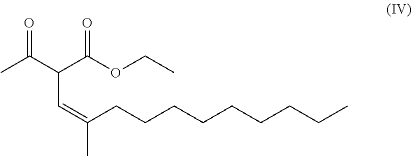 [SMILES: CCCCCCCCC/C(C)=C\C(C(C)=O)C(=O)OCC]

Ethyl 2-acetyl-4-methyltridec-3-enoate (III) was obtained by repeated chromatographic purification of a sample (9.0 g) of the crude product obtained from the condensation of ethyl acetoacetate and 2-methyl undecanal as described in Example 1, which had been stored in a closed bottle at 50° C. for 1 month. The sample was first chromatographed over SiO₂ with hexane/MTBE 4:1 to afford ca. 3 g of a light yellow oil. This material was then chromatographed over A103 with hexane/MtBE 4:1 to give 1.2 g of a colorless and virtually odourless liquid. The NMR-spectra reveal the presence of a 57:43 mixture of enol and keto forms, both as E-/Z-mixtures.

¹H-NMR (400 MHz, C₆D₆; mixture of enol and keto forms as described above) 13.48 (q, J=0.7 Hz, 0.4H), 13.46 (q, J=0.7 Hz, 0.2H), 5.78 (br. s, J=0.7 Hz, 0.5H), 5.62-5.73 (series of m, 0.8H), 4.35 (d, J=9.8 Hz, 0.2H), 4.26 (d, J=9.3 Hz, 0.2H), 3.90-4.02 (series of m, 2H), 1.87 2.06 (series of m, 1.7H), 1.92 (s, 0.8H), 1.90 (s, 1H), 1.79-1.83 (m, 1.7H), 1.68 (d, J=1.5 Hz, 0.7H), 1.59 (d, J=1.5 Hz, 0.6H), 1.47 (d, J=1.5 Hz, 0.7H), 1.43 (d, J=1.5 Hz, 1.3H), 1.11-1.35 (m, 14H), 0.86-1.00 (series of m, 6H).

¹³C-NMR (100 MHz, C₆D₆; mixture of enol and keto forms as described above) 200.7 (s), 200.5 (s), 173.8 (s,), 173.7 (s), 173.7 (s), 173.7 (s), 169.4 (s), 169.4 (s), 142.3 (s), 142.1 (s), 141.1 (s), 140.8 (s), 118.8 (d), 118.5 (d), 118.0 (d), 117.5 (d), 100.2 (s), 99.9 (s), 61.5 (t), 61.5 (t), 60.8 (t), 60.7 (t), 60.6 (d), 60.5 (d), 40.2 (t), 39.8 (t), 33.3 (t), 32.9 (t), 32.7 (t), 30.5 (t), 30.4 (t), 30.4 (t), 30.3 (t), 30.2 (t), 30.2 (t), 29.9 (t), 28.7 (t), 28.5 (t), 28.4 (t), 27.9 (t), 27.8 (t), 23.8 (q), 23.5 (t), 22.9 (q), 19.9 (q), 17.9 (q), 16.9 (q), 14.7 (q), 14.6 (q), 14.4 (q).

### Example 4: Preparation of dimethyl 2-(2-methylundecylidene)malonate (V)

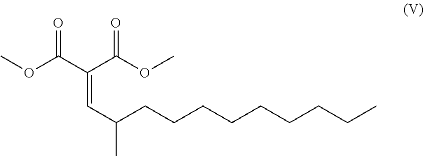 [SMILES: CCCCCCCCCC(C)C=C(C(=O)OC)C(=O)OC]

A solution of dimethyl malonate (16.5 g, 125 mmol, 1.15 equiv.), 2-methyl undecanal (20.0 g, 109 mmol, 1.0 equiv.), acetic acid (0.98 g, 16.3 mmol, 0.15 equiv.) and piperidine (0.46 g, 5.4 mmol, 0.05 equiv.) in cyclohexane (180 mL) was heated to reflux under stirring for 22 h in a round-bottomed flask equipped with a Dean-Stark apparatus. After cooling to room temperature, the mixture was diluted with MTBE and washed with water and brine. The organic layer was separated, dried over MgSO₄ and concentrated under reduced pressure in a rotary evaporator to yield a clear, slightly yellow liquid (32.5 g, <99%), which exhibited a fruity-aldehydic smell.

¹H-NMR (400 MHz, C₆D₆) 6.80 (d, J=10.8 Hz, 1H), 3.82 (s, 3H), 3.78 (s, 3H), 2.46-2.59 (m, 1H), 1.25 (m, 16H), 1.05 (d, J=6.6 Hz, 3H), 0.88 (t, J=6.6 Hz, 3H).

¹³C-NMR (100 MHz, C₆D₆) 166.0 (s), 164.4 (s), 155.3 (d), 126.6 (s), 52.2 (q), 52.1 (q), 36.4 (t), 34.7 (d), 31.8 (t), 29.5 (t), 29.4 (t), 29.3 (t), 27.2 (t), 26.9 (t), 22.6 (t), 19.8 (q), 14.0 (q).

### Example 5: Preparation of isopropyl 2-acetyl-4-methyltridec-2-enoate (VI)

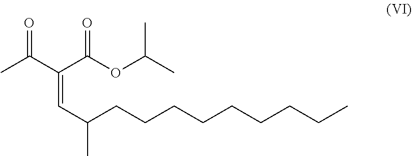 [SMILES: CCCCCCCCCC(C)/C=C(/C(C)=O)C(=O)OC(C)C]

A mixture of isopropyl 3-oxobutanoate (20.0 g, 139 mmol, 1.05 equiv.), 2-methyl undecanal (24.4 g, 132 mmol, 1.0 equiv.), and piperidine (1.1 g, 13.2 mmol, 0.1 equiv.) was left to stand in a round bottomed flask at room temperature for 2 days, then diluted with MTBE (100 mL) and washed with diluted aq. NaCl-solution. The organic layer was separated, dried over MgSO₄ and concentrated under reduced pressure in a rotary evaporator to yield a clear, yellow liquid (36.4 g, 89%), which exhibited a fruity-aldehydic smell.

¹H-NMR (400 MHz, CDCl₃, mixture of E- and Z-isomers) 6.65 (d, J=11.0 Hz, 0.36H), 6.57 (d, J=10.8 Hz, 0.64H), 5.20 (hept, J=6.3 Hz, 0.64H), 5.12 (hept, J=6.4 Hz, 0.36H), 2.60-5.42 (m, 1H), 2.35 (s, 1H), 2.31 (s, 2H), 1.35-1.23 (series of m, 22H), 1.07 (d, J=6.6 Hz, 2H), 1.04 (d, J=6.6 Hz, 1H), 0.86-0.92 (br. t, J=7.1 Hz, 3H).

¹³C-NMR (100 MHz, CDCl₃, mixture of E- and Z-isomers) 201.4 (s), 195.1 (s), 166.3 (s), 164.1 (s), 153.3 (d), 152.9 (d), 136.0 (s), 134.9 (s), 68.9 (d), 68.8 (d), 36.6 (t), 36.5 (t), 34.8 (d), 34.0 (d), 31.9 (t), 29.6 (t), 29.6 (t), 29.5 (t), 29.3 (t), 27.4 (t), 27.0 (q), 26.9 (q), 22.7 (t), 21.8 (q), 20.1 (q), 19.9 (q), 14.1 (q).

### Example 6: Preparation of ethyl 4-methyl-2-propionyltridec-2-enoate (VII)

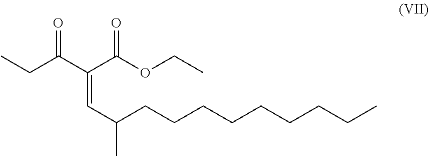 [SMILES: CCCCCCCCCC(C)/C=C(/C(=O)CC)C(=O)OCC]

The procedure described in Example 5 was repeated with ethyl 3-oxopentanoate (10.0 g, 69 mmol, 1.05 equiv.), 2-methyl undecanal (12.2 g, 66 mmol, 1.0 equiv.) and piperidine (0.56 g, 6.6 mmol, 0.1 equiv.) to yield a clear, yellow liquid (20.5 g, >99%), which exhibited a fruity-aldehydic smell.

¹H-NMR (400 MHz, CDCl₃, mixture of E- and Z-isomers) 6.66 (d, J=11.0 Hz, 0.46H), 6.58 (d, J=10.5 Hz, 0.54H), 4.30 (q, J=7.1 Hz, 1.2H), 4.23 (q, J=7.1 Hz, 0.8H), 2.58-2.72 (m, 2H), 2.27-2.42 (m, 1H), 1.21-1.39 (m, 19H), 1.01-1.15 (m, 6H), 0.88 (br. t, J=Hz, 3H).

¹³C-NMR (100 MHz, CDCl₃, mixture of E- and Z-isomers) 204.6 (s), 197.9 (s), 166.9 (s), 164.7 (s), 153.0 (d), 152.2 (d), 135.3 (s), 134.4 (s), 61.1 (t), 61.1 (t), 37.0 (t), 36.6 (t), 36.5 (t), 34.8 (d), 34.2 (d), 32.3 (t), 31.9 (t), 29.6 (t), 29.6 (t), 29.6 (t), 29.5 (t), 27.4 (t), 27.4 (t), 22.7 (t), 20.2 (q), 19.9 (q), 14.2 (q), 14.1 (q), 14.1 (q), 13.3 (q), 8.0 (q), 7.8 (q).

### Example 7: Preparation of ethyl 2-cyano-4-methyltridec-2-enoate VIII)

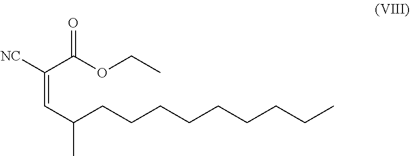 [SMILES: CCCCCCCCCC(C)/C=C(/C#N)C(=O)OCC]

The procedure described in Example 1 was repeated with ethyl 2-cyanoacetate (11.3 g, 100 mmol, 1.0 equiv.), 2-methyl undecanal (18.4 g, 100 mmol, 1.0 equiv.) and piperidine (0.17 g, 2.0 mmol, 0.02 equiv.), and the mixture was left to stand at room temperature for 64 h, then workup was effected as described in Example 6 to yield a clear, yellow liquid (25.7 g, 92%), which exhibited a fruity-aldehydic smell.

¹H-NMR (400 MHz, CDCl₃) 7.44 (d, J=10.8 Hz, 1H), 4.32 (q, J=7.3 Hz, 2H), 2.78-2.96 (m, 1H), 1.36 (t, J=7.1 Hz, 3H), 1.18-1.57 (series of m, 16H), 1.14 (d, J=6.8 Hz, 3H), 0.88 (br t, J=6.4 Hz, 3H).

¹³C-NMR (100 MHz, CDCl₃) 168.7 (d), 161.4 (s), 113.7 (s), 108.3 (s), 62.4 (t), 37.1 (d), 36.0 (t), 31.8 (2 t), 29.5 (t), 29.4 (t), 29.3 (t), 27.3 (t), 22.7 (t), 19.4 (q), 14.1 (2 q).

### Example 8: Improvement of Thermal Stability by Purification of the Product (Thermal Stability of (III) with and without Chromatographic Purification)

Rac. ethyl 2-acetyl-4-methyltridec-2-enoate (III) was prepared according to Example 1 with the difference that instead of 0.4 mol %, only 0.2 mol % of piperidine was employed. A sample of the crude material (20.0 g) was purified by silica gel column chromatography with hexane/MTBE 4:1 as eluent. The impurities were removed and all isomers of ethyl 2-acetyl-4-methyltridec-2- and 3-enoate were recombined and dried in vacuum to yield 10.0 g (50%) of a pale yellow oil.

The composition of the crude and chromatographed material was determined by ¹H-NMR spectroscopy with internal standard (p-methoxy benzaldehyde) at T₀ and after a storage time of 2 weeks at 50° C. in a closed glass bottle (ca. 5 g samples).

| | T₀ | | | | | after 2 weeks at 50° C. | | | | |
|---|---|---|---|---|---|---|---|---|---|---|
| [wt-%] in | | | | Z:E | Active | | | | Active | Z:E |
| mixture | Z-(III) | E-(III) | (IV) | ratio | Precursor | Z-(III) | E-(III) | (IV) | Precursor | ratio |
| Crude Product | 49 | 30 | 7 | 79 | 1.6 | 24 | 12 | 50 | 36 | 2 |
| Purified Product | 53 | 33 | 7 | 86 | 1.6 | 47 | 9 | 37 | 56 | 5.2 |

The results show that chromatographic purification results in a decreased thermal double bond shift rate. They also show that in the purified product, devoid of certain impurities (e.g. piperidine derivatives), which are capable of catalyzing the Z- to E-isomerization, the double bond shift occurs faster from the E-isomer resulting in an increase of the Z- to E-ratio from 2 to 5.2 after the storage period. This example illustrates the advantage of a chromatographic purification of the product for improved thermal stability.

### Example 9: Improvement of Thermal Stability by Using the Z-Rather than the E-Isomer of the Precursor

The composition of pure E- and Z-isomers of rac. ethyl 2-acetyl-4-methyltridec-2-enoate (as described in example 2) were determined at T₀ and after a storage time of 1 week at 50° C. in a closed glass bottle (ca. 5 g samples). The quantification was carried out by spectroscopy with internal standard (p-methoxy benzaldehyde).

| | T₀ | | | | after 1 week at 50° C. | | | |
|---|---|---|---|---|---|---|---|---|
| [%] in | | | | Active | | | | Active |
| mixture | Z-(III) | E-(III) | (IV) | Precursor | Z-(III) | E-(III) | (IV) | Precursor |
| E-isomer | 0 | 97 | 1 | 97 | 23 | 15 | 47 | 38 |
| Z-isomer | 98 | 0 | 0 | 98 | 78 | 12 | 4 | 90 |

These surprising results show that after 1 week at 50° C., the active precursor content was still 90% when starting with the Z-isomer and only 38% when starting with the E-isomer. This example illustrates the advantage of a product composition with a high Z-content for improved thermal stability, provided the product is devoid of impurities capable of catalyzing the Z- to E-isomerization.

### Example 10: Improvement of Thermal Stability by Avoiding Impurities which Catalyze Z- to E-Isomerization

The results detailed in Examples 8 and 9 clearly suggest that for compounds of generic formula (I) with X=—COR and Y=—CO₂R′, it is desirable to preserve the stereochemical integrity of the Z-isomer during storage so that it will not convert into the faster decomposing E-isomer. In order to test which impurities present from the manufacturing process are capable of catalyzing the undesired Z- to E-isomerization and thus should be avoided, a number of chemicals were admixed at various concentrations to pure ethyl (Z)-2-acetyl-4-methyltridec-2-enoate Z-(III). The resulting mixtures were stored in closed vials at 50° C. for 24 h and the E-/Z-isomer ratio was determined by spectroscopy.

The following table illustrates the outcome of this experiment when 2-methyl undecanal was admixed to pure ethyl (Z)-2-acetyl-4-methyltridec-2-enoate Z-(III):

| | |
|---|---|
| | Composition after |
| Amount of 2-methyl undecanal | 24 h at 50° C. [mol %] |

| admixed to pure Z-(III) [vol %] | Z-(III) | E-(III) |
|---|---|---|
| 0.001 | 100 | 0 |
| 0.01 | 100 | 0 |
| 0.1 | 100 | 0 |
| 1 | 96 | 4 |
| 10 | 64 | 36 |

The results show that the stereochemical integrity of the Z-precursor isomer is affected when ≥1% of 2-methyl undecanal were added. Thus, a product containing less than 1%, preferably less than 0.1% of free aldehyde will exhibit a better thermal stability and is preferred.

Similarly, the presence of amines, such as piperidine or piperazine, were found to promote Z- to E-isomerization and therefore have to be kept <0.1%, preferably >0.01%.

Free ethyl acetoacetate should be kept below 10%, preferably below 5% and even more preferably below 1%.

### Example 11: Improvement of Thermal Stability by Modifying the 1,3-Dicarbonyl Unit

The experiment described in 9 was repeated with compounds (I) of the current invention with varying X and Y groups. The loss of active precursor after 1 week at 50° C. is reported in the below table.

| | | Loss of active precursor after 1 week at 50° C. |
|---|---|---|
| X | Y | [wt %] |
| —COMe | —CO₂Et | 40 |
| —COMe | —CO₂iPr | 33 |
| —COEt | —CO₂Et | 24 |
| —CO₂Me | —CO₂Me | 4 |
| —CN | —CO₂Et | 1 |

These results show that the rate of thermal degradation of the product strongly depends on the nature of the groups X and Y and that the proper choice of those groups will lead to products with improved thermal stability.

As can be seen from these results, the diester and the cyanoester are more stable than the keto-esters.

### Example 12: Improvement of Stability by Storing the Product at Low Temperature

Samples of ethyl 2-acetyl-4-methyltridec-2-enoate (III) prepared according to Example 1 were stored at 24° C. and at 4° C. The loss of active precursor (ethyl Z- and E-2-acetyl-4-methyltridec-2-enoate) was measured by the method described in Example 9. Whereas at 24° C. an average loss of 2-3 mol %/week was recorded, at 4° C. the loss was only 0.2 mol %/week. This result exemplifies the advantage of storing the precursor in the cold, preferably below 20° C., more preferably below 10° C. and even more preferably between 0-5° C. after its manufacture until it is incorporated in the targeted consumer product.

1. A precursor compound of a fragrant aldehyde according to the formula (I) 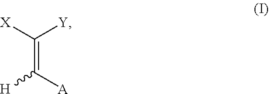
wherein:
A is a hydrocarbon residue of a fragrant aldehyde A-CHO, wherein the hydrocarbon residue may optionally contain one or more hetero-atom(s) selected from O, N, S and/or Si;
X and Y are independently selected from the group consisting of: a nitrile (—CN), a keto (—COR), and an ester (—CO₂R′) functional group, wherein R and R′ are independently an alkyl residue selected from: methyl, ethyl, propyl, isopropyl, butyl, sec-butyl, iso-butyl and pentyl, provided that:
i) X and Y cannot both represent keto residues; and
ii) when X and Y represent different functional groups, wherein one group is an ester group and the other one is a keto group, the alkylidene double bond is enriched in its Z-isomer.

2. The compound according to claim 1, according to the following formula (II) 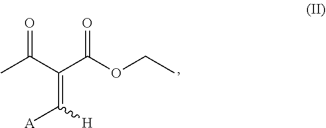
wherein the akylidene double bond is enriched in its Z-isomer.

3. The compound according to claim 1 being ethyl 2-acetyl-4-methyltridec-2-enoate according to the following formula (III) 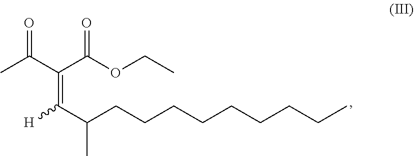 [SMILES: CCCCCCCCCC(C)C=C(C(C)=O)C(=O)OCC]
wherein the alkylidene double bond is enriched in its Z-isomer.

4. Compounds according to formula (I) of claim 1, wherein the Z:E ratio of the compounds is 55:45 to 100:0.

5. Compounds according to claim 4, which are free or essentially free of the un-conjugated isomer according to the following formula (IV)

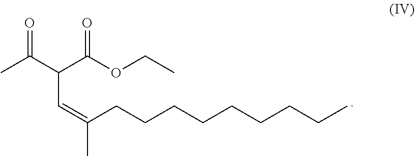 [SMILES: CCCCCCCCC/C(C)=C\C(C(C)=O)C(=O)OCC]

6. A method of forming ethyl 2-acetyl-4-methyltridec-2-enoate enriched in its Z-isomer, said method comprising the step of: carrying out a Knoevenagel condensation reaction between ethyl acetoacetate and 2-methyl undecanal in the presence of a base.

7. The method of claim 6, comprising the step of: removing residual base and/or residual derivatives of the base.

8. The method according to claim 6, wherein the base is a piperazine or a piperidine.

9. The method according to claim 6, comprising the step of: removing residual unreacted 2-methyl-undecanal from the reaction mixture.

10. The method according to claim 6, comprising the step of: storing ethyl 2-acetyl-4-methyltridec-2-enoate (III) at a temperature of about 20° C. or less.

11. A kit comprising a compound according to formula (I) of claim 1 in a container, with labeling, instructions and/or packaging providing instructions to store the container containing said compound at a temperature of about 20° C. or less.

12. A method of imparting the fresh odour of a fragrant aldehyde A-CHO to a dry or drying situs, comprising the steps of: treating the situs with a laundry care composition, a personal care composition, or a household care composition containing a compound (I) according to claim 1, and allowing the situs to dry.

13. The method according to claim 12, comprising the step of: washing or treating fabric with the laundry care composition containing said compound (I), and thereafter allowing the fabric to dry.

14. The method according to claim 12, comprising the step of of: washing or treating human hair or skin with the personal care composition containing said compound (I), and thereafter allowing the hair or skin to dry.

15. The method according to claim 12, comprising the step of: applying the household surface cleaner containing said compound (I) or treatment composition containing said compound (I), to a household surface, and allowing the surface to dry.

16. Laundry care, personal care r household care compositions comprising the compound of formula (I) according to claim 1.

17. The method according to claim 13, wherein the washing or treating the said fabric with the laundry care composition takes place in an aqueous liquor containing a detergent, or fabric conditioner, or fabric treatment composition containing said compound (I), and thereafter allowing the fabric to dry.

18. The method according to claim 12, wherein the personal care composition is selected from a hair care, body care, or cosmetic product.
